(19) United States
(12) Patent Application Publication
(10) Pub. No.: US 2024/0270904 A1
(43) Pub. Date: Aug. 15, 2024

(54) METHOD FOR NANOMORPHOLOGICAL MODIFICATION OF POLYETHER ETHER KETONE SURFACE BY HOT PRESSING, MODIFIED POLYETHER ETHER KETONE MATERIAL AND APPLICATION THEREOF

(71) Applicant: AIR FORCE MEDICAL UNIVERSITY, Xi'an (CN)

(72) Inventors: Liang KONG, Xi'an (CN); Fuwei LIU, Xi'an (CN); He XIN, Xi'an (CN); Weiwei WU, Xi'an (CN); Gandong ZHOU, Xi'an (CN); Xuelian JIA, Xi'an (CN); Yunpeng LI, Xi'an (CN); Bolei CAI, Xi'an (CN); Lei TIAN, Xi'an (CN); Mingchao DING, Xi'an (CN); Ye GAO, Xi'an (CN); Yan HOU, Xi'an (CN); Qianxin LV, Xi'an (CN)

(21) Appl. No.: 18/411,739

(22) Filed: Jan. 12, 2024

(30) Foreign Application Priority Data
Feb. 13, 2023 (CN) 202310103870.0

(51) Int. Cl. C08G 65/40; A61C 8/00; B29C 43/00; B29K 71/00; B29L 31/00; C08K 3/36

(52) U.S. Cl. CPC C08G 65/4012; A61C 8/0016; B29C 43/003; C08K 3/36; B29K 2071/00; B29L 2031/753; C08G 2650/40; C08K 2201/003; C08K 2201/011

(57) ABSTRACT

Disclosed is a method for nanomorphological modification of polyether ether ketone (PEEK) surface by hot pressing, relating to the technical field of biomaterials. The method includes the following steps: S1, preparing SiO2 nanospheres; S2, uniformly laying the SiO2 nanospheres prepared in the S1 on a surface of a PEEK material; S3, heating the surface of the PEEK material to a temperature higher than a glass phase transition temperature, then carrying out hot pressing to embed the SiO2 nanospheres into the surface of the PEEK material, and naturally cooling at room temperature after the hot pressing; S4, after cooling, using NaOH solution to corrode and remove the SiO2 nanospheres on the surface of the PEEK material after hot pressing in S3 to form a structure with nanoconcaves, then obtaining a hot-pressed modified PEEK material.

### CROSS-REFERENCE TO RELATED APPLICATIONS

This application claims priority to Chinese Patent Application No. 202310103870.0, filed on Feb. 13, 2023, the contents of which are hereby incorporated by reference.

### TECHNICAL FIELD

The present application relates to the technical field of biomaterials, in particular to a method for nanomorphological modification of polyether ether ketone (PEEK) surface by hot pressing, a modified PEEK material and application thereof.

### BACKGROUND

Polyether ether ketone (PEEK) materials are semi-crystalline organic polymers that have received increasing attention of scholars because of their excellent corrosion resistance, biocompatibility, minimal interference with imaging, and mechanical properties close to natural bone tissue. Since 1992, PEEK has been employed in field of dentistry, initially as a material based on its aesthetic restorative benefits, and later with more application in jaw implant placement. The growing popularity of PEEK among clinicians is also based on the following advantages:
chemical stability: PEEK materials are corrosion resistant, chemically stable, and inert in vivo; experiments on its biosafety have confirmed that PEEK materials are free of cellular, tissue, or genetic toxicity, while in vitro and in vivo experiments have confirmed that PEEK materials are well compatible with a variety of cells and tissues such as epithelium, muscle, bone, and cartilage;
favorable mechanical properties: the biomechanical parameters of the PEEK material are more similar to those of cortical bone (16-23 GPa, and 80-150 MPa respectively) in comparison to titanium and titanium alloys (110 GPa; 897-1034 MPa), with which the “stress shielding” effect is avoided and biomechanical microenvironment of cells may be simulated in vivo; and
no interference with imaging examinations: the PEEK material presents a transmissive shadow in clinical radiographs, thus preventing artifacts on X-rays and computed tomography (CT) examinations, scattering artifacts caused by magnetic resonance imaging (MRI), and heat generation from the implant, making it a valuable tool for joint surgery and neurosurgery; in contrast, PEEK material is white in color, which makes it well suited for aesthetic medical areas such as the anterior teeth of the mouth; also, PEEK material does not generate heat during high-speed rotational contact with the cutting drill, which will facilitate temporary adjustments and intraoperative shaping of the implant during clinical surgical operations, increasing the tolerance and applicability of the implant in clinical use. As such, PEEK is considered an ideal alternative material to conventional titanium bone implants and has been increasingly used as one of the emerging options for fracture fixation and bone defect reconstruction and repair in the clinical application in recent years, especially in the developed countries of Europe and America.

However, it is found in practice that the PEEK material is too inert and lacks bioactive sites to develop effective osseointegration with the surrounding bone tissue. After implantation, it relies only on screw fixation, which causes high local stresses and high mechanical risks such as screw dislodgement and local fracture in long-term application, therefore seriously limits the application and promotion of this method in the clinical treatment of jaw defects and deformities.

Accordingly, the bioactivity of PEEK materials can be improved by surface modification techniques, with little change in mechanical properties. Surface modification techniques involve three main categories: physical morphology optimization, chemical modification, and biological modification.

As conventional strategies to improve the interaction effect between biomaterials and surrounding tissues, surface modification techniques involve three main categories: physical morphology optimization, chemical modification, and biological modification. Physical morphology optimization is one of the surface treatment techniques, initially used as an important method to increase interfacial roughness, mechanical embedding force, tissue contact area, and wettability, etc. It is a mature and controllable process and is considered to be an effective method to promote interaction between implants and surrounding tissues, which has been widely recognized and applied in clinical practice. Chemical modification is an important method to increase the biological activity of implant materials, including primarily loading the implant surface with chemical groups such as hydroxyl and amino groups that are biologically active or chemical elements and substances such as strontium and hydroxyapatite that have pro-cellular functions. Biological modification mainly involves the integration of biologically active molecules such as bone morphogenetic protein (BMP) and collagen on the surface of the implant; yet, the complex functional mechanism of biologically active molecules remains largely to be investigated, and the treatment process is far from developed. Similar to chemical modification, the controlled releasing, safety and clinically validation of the added components of biological modification require to be further studied, making productization and clinical promotion rather difficult. Thus, to meet the requirements of safety, productization, and scalability for clinical applications, the physical morphology modification of the surface is preferred. On this basis, it is expected to prepare a physical structure on the PEEK surface that conforms to natural tissue properties in order to enhance the interaction between the PEEK material and the surrounding tissues solely from a bionic perspective, which will help promote PEEK osseointegration.

Nanomorphology has gained increasing attention for its ability of imitating the nanostructured units in natural tissues thanks to the deepened understanding of the microstructure of natural bone tissue, and the research, construction and application of nanomorphology on the surface of biomaterials have gradually become hot spots in the industry with the continuous development of processing technology and detection technology. Currently, there are available methods including anodic oxidation, plasma spraying, magnetron sputtering, and electrodeposition to prepare structures such as nanotubes, nanofibers, nanoconcaves, and nanopillars. Studies have shown that nanomorphological surface wettability, surface energy and roughness can imitate extracellular matrix proteins such as fibronectin and vitronectin, increasing cell adhesion and inducing changes in intracellular organelles or key molecules by affecting cytoskeletal arrangement, and exert an impact on early behavioral aspects of cell migration together with adhesion proteins. Nanostructures will also induce different biological responses by affecting the arrangement and translocation of receptors and related proteins on the cell membrane, such as directly promoting osteogenic differentiation of bone marrow mesenchymal stem cells (BMMSCs), enhancing the expression and secretion of key osteogenic molecules such as runt-related transcription factor 2 (Runx2), alkaline phosphatase (ALP), osteopontin (OPN), and osteocalcin (OCN), etc. It is also demonstrated in animal experiments that nanomorphological surfaces can significantly increase new bone formation and osseointegration of bone implants. In addition, nanomorphology promotes adhesion of fibroblasts, enhances the expression and secretion of adhesion-related proteins such as Vinculin and Integrin, and promotes their binding to soft tissues.

To achieve nanomorphology optimization on the surface of PEEK materials, there are two main types of methods available:
acid etching: the prevailing method; PEEK is highly resistant to corrosion, so it is generally acid etched with concentrated sulfuric acid, which dissolves the PEEK surface and forms an irregular shape by “subtraction”; this preparation method is very efficient, but it will inevitably cause loosening of the PEEK fibers and affect the strength of the interface; besides, it is difficult to accurately regulate the preparation parameters; moreover, acid etching is not a simple morphological modification method as sulfonic acid groups are introduced after sulfonation; at present, it is mainly used as a means to achieve roughening and increase contact area; and
surface coating modification: there are mainly five methods of coating other substances onto the surface of PEEK, including: plasma spraying, cold spraying, aerosol deposition, electron beam deposition, and rotary coating, etc., all of which involve the loading of metallic or non-metallic coatings onto the surface of the material substrate in the “addition” manner; although these methods are simple for preparation, the weak bonding between the coating and the PEEK substrate is the main problem that limits the application of this method, and the fine structure shaping force is not strong enough.

To address these issues, there is an urgent need to develop a surface modification method that does not change the chemical composition of the PEEK material surface and does not introduce exogenous substances, but achieves a complete nanomorphological modification of the PEEK material surface and effectively promotes the bonding between the PEEK material and the hard and soft tissues of the bone implant site.

### SUMMARY

In order to overcome the core problems of the above methods, namely the introduction of exogenous groups, the lack of mechanical strength in the morphology optimization area and the lack of nanoscale fine structure shaping force, the present application provides a method for nanomorphological modification of polyether ether ketone (PEEK) surface by hot pressing; using the “hot pressing method”, a nano-concave morphology of uniform and controllable diameter is prepared on the surface of PEEK material. The objectives of the present application are achieved by the technical schemes as follows:
a method for nanomorphological modification of PEEK surface by hot pressing, including steps as follows:
   S1, preparing SiO₂ nanospheres;
   S2, uniformly laying the SiO₂ nanospheres prepared in the S1 on a surface of a PEEK material;
   S3, heating the surface of the PEEK material to a temperature higher than a glass phase transition temperature, then carrying out hot pressing to embed the SiO₂ nanospheres into the surface of the PEEK material, and naturally cooling at a room temperature after the hot pressing; and
   S4, after cooling, using NaOH solution to corrode and remove the SiO₂ nanospheres on the surface of the PEEK material after hot pressing of S3 to form a structure with nanoconcaves, and then obtaining a hot-pressed modified PEEK material.

Optionally, the S1 includes specific operations as follows:
using ethyl orthosilicate as a silicon source and aqueous ammonia as a catalyst, preparing monodispersed SiO₂ nanospheres by a sol-gel method, where a diameter of the SiO₂ nanospheres is 100-1,000 nanometers (nm).

Optionally, a mass fraction of the ethyl orthosilicate is 28%, a mass fraction of the aqueous ammonia is 25%-28%; and the sol-gel method includes adding ethanol, water, aqueous ammonia and tetraethyl orthosilicate in a volume ratio of 6:6:1:(1-10), and reacting for a duration of 2-24 hours (h).

Optionally, specific operations of the S2 includes steps as follows:
S21, preparing the SiO₂ nanospheres prepared in the S1 into an ethanol solution of the SiO₂ nanospheres with a mass fraction of 5%-10%;
S22, cleaning the PEEK material with acetone, ethanol and deionized water in turn; and
S23, performing film-drawing using the ethanol solution of the SiO₂ nanospheres obtained in S21 on the PEEK material after cleaning by using a Langmuir-Blodgett (LB) film-drawing machine.

Optionally, in the S3, a temperature for the hot pressing is 140-160 degrees Celsius (° C.), an air pressure of the hot pressing is 0.5-0.8 megapascal (MPa), and a duration for the hot pressing is 15-20 minutes (min).

Optionally, a concentration of the NaOH solution in S4 is 5-7 molality (M).

A hot-pressed modified PEEK material obtained by the above method for nanomorphological modification of PEEK surface by hot pressing.

Optionally, a structure of nanoconcaves on a surface of the hot-pressed modified PEEK material has a diameter of 100-1,000 nm and a depth of 50-500 nm.

An application of the hot-pressed modified PEEK material as a bone implant.

Compared with the prior art, the present application achieves the beneficial effects that:
the present application achieves pure nanomorphological modification of the surface of PEEK materials without altering the chemical composition of the PEEK material and without introducing exogenous substances, and effectively promotes the osseointegration properties of PEEK materials with high biosafety, thereby providing a basis for clinical applications;
the method of the present application makes the PEEK material surface fibers denser by hot pressing during the preparation of nanomorphology, introducing no coating and eliminating the separation of coating from the substrate, thus solving the problem of insufficient mechanical strength in the morphology optimization area; and
according to the method of the present application, the desired nanomorphology may be finely regulated by adjusting the diameter of SiO₂ nanospheres and the pressurized depth, and the fine structure of nanoconcaves with fixed diameter and depth is prepared on the surface of PEEK implant, which is similar to the precision of the nanotube structure prepared by the classical anodic oxidation method on the surface of titanium alloy, thus solving the problem of insufficient shaping force of the nanoscale fine structure.

### BRIEF DESCRIPTION OF THE DRAWINGS

FIG. 1 is a schematic diagram of a method for nanomorphological modification of polyether ether ketone (PEEK) surface by hot pressing and application thereof.

FIG. 2 shows a process of the method for nanomorphological modification of PEEK surface by hot pressing according to an embodiment of the present application.

FIG. 3 shows scanning electron microscopy (SEM) images of surfaces of hot-pressed modified PEEK materials.

FIG. 4A is a statistical diagram of the hot-pressed modified PEEK materials at a diameter of 100 nm.

FIG. 4B is a statistical diagram of the hot-pressed modified PEEK materials at a diameter of 500 nm.

FIG. 4C is a statistical diagram of the hot-pressed modified PEEK materials at a diameter of 1000 nm.

FIG. 5A is an AFM image of the surfaces of the hot-pressed modified PEEK materials at Peek 0.

FIG. 5B is an AFM image of the surfaces of the hot-pressed modified PEEK materials at Peek 100.

FIG. 5C is an AFM image of the surfaces of the hot-pressed modified PEEK materials at Peek 500.

FIG. 5D is an AFM image of the surfaces of the hot-pressed modified PEEK materials at Peek 1000.

FIG. 6 is the result of roughness test on the surface of hot-pressed modified PEEK materials.

FIG. 7A is a Fourier infrared detection diagram of the surfaces of the hot-pressed modified PEEK materials.

FIG. 7B is an X-ray diffraction inspection diagram of the surfaces of the hot-pressed modified PEEK materials.

FIG. 8A is an elastic modulus test plot of the surfaces of hot-pressed modified PEEK materials.

FIG. 8B is a nano-hardness test plot of the surfaces of hot-pressed modified PEEK materials.

FIG. 9 shows results of water contact angle testing on the surfaces of hot-pressed modified PEEK materials.

FIG. 10 shows observation diagrams of rat bone marrow stromal cells (rBMSCs) cultured on the surfaces of the hot-pressed PEEK materials after 4′,6-diamidino-2′-phenylindole (DAPI) staining.

FIG. 11 shows observation diagrams of human skin fibroblast (HSF) cells cultured on the surfaces of the hot-pressed PEEK materials after DAPI staining.

FIG. 12 shows immunofluorescence observation diagrams of the HSF cells cultured on the surfaces of the hot-pressed PEEK materials after DAPI staining.

FIG. 13A is an image of immunofluorescence staining of the macrophage M1 polarization indicator iNOS as well as the M2 polarization indicator Arg-1 on the surface of hot-pressed PEEK material.

FIG. 13B illustrates a quantitative analysis of immunofluorescence staining for imacrophage M1 polarization indicator iNOS on the surface of hot-pressed PEEK material.

FIG. 13C illustrates a quantitative analysis of immunofluorescence staining for imacrophage M1 polarization indicator iNOS as well as the M2 polarization indicator Arg-1 on the surface of hot-pressed PEEK material.

FIG. 14 is an assay of NO release from macrophages inoculated on the surfaces of hot-pressed modified PEEK materials for 6 hours.

FIG. 15 shows the results of cell scratching experiments of human umbilical vein endothelial cells (HUVEC) inoculated on the surfaces of the hot-pressed modified PEEK materials.

FIG. 16 shows a quantitative analysis of the scratch healing rate of surface scratching experiments on the surfaces of the hot-pressed modified PEEK materials.

FIG. 17A is an experimental image of HUVEC tube formation assay on the surface of the hot-pressed PEEK material.

FIG. 17B is a quantitative analysis of the total number of blood vessel branches in HUVEC tube formation assay on the surface of the hot-pressed PEEK material.

FIG. 17C is a quantitative analysis of the total length of blood vessels formed by HUVEC tube on the surface of the hot-pressed PEEK material.

FIG. 17D is a quantitative analysis of the percentage of blood vessel area formed by HUVEC tube on the surface of the hot-pressed PEEK material.

FIG. 18A is an image of confocal detection of VEGF immunofluorescence staining of HUVEC and macrophages co-cultured on the surface of hot-pressed PEEK material.

FIG. 18B is a quantitative analysis of VEGF immunofluorescence staining of HUVEC and macrophages co-cultured on the surface of hot-pressed PEEK material

FIG. 19A shows the osteogenic differentiation indicator ALP (7 days, 14 days) and alizarin red stained (14 days, 21 days) images of the surface of the hot-pressed PEEK material.

FIG. 19B is a quantitative analysis of ALP, an indicator of osteogenic differentiation, on the surface of the hot-pressed PEEK material.

FIG. 19C illustrates a quantitative analysis of osteogenic differentiation on the surface of the hot-pressed PEEK material: alizarin red staining.

FIG. 20A is an osteogenic micro-CT reconstructed image of the side of the implant in a rabbit skull 8 weeks after implantation of a hot-pressed PEEK implant.

FIG. 20B is a quantitative analysis of the bone volume ratio (BV/TV) on the side of the implant in the rabbit skull 8 weeks after implantation of the hot-pressed PEEK implant.

FIG. 20C is a quantitative analysis of trabecular thickness (TbTh) of bone on the side of the implant in the rabbit skull 8 weeks after implantation of the hot-pressed PEEK implant.

FIG. 20D is a quantitative analysis of the number of bone trabecular (TbN) on the side of the implant in the rabbit skull 8 weeks after implantation of the hot-pressed PEEK implant.

FIG. 20E is a quantitative analysis of trabecular spacing (TbSp) on the side of the implant in the rabbit skull 8 weeks after implantation of the hot-pressed PEEK implant.

FIG. 21A is an osteogenic micro-CT reconstructed image of the top surface of the implant in a rabbit skull 8 weeks after implantation of the hot-pressed PEEK implant.

FIG. 21B is a quantitative analysis of the bone volume ratio (BV/TV) of the top surface of the implant in the rabbit skull 8 weeks after implantation of the hot-pressed PEEK implant.

FIG. 21C is a quantitative analysis of trabecular thickness (TbTh) of bone on the top surface of the implant in the rabbit skull 8 weeks after implantation of the hot-pressed PEEK implant.

FIG. 21D is a quantitative analysis of the number of bone trabecular (TbN) on the top surface of the implant in the rabbit skull 8 weeks after implantation of the hot-pressed PEEK implant.

FIG. 21E is a quantitative analysis of trabecular spacing (TbSp) on the top surface of the implant in the rabbit skull 8 weeks after implantation of the hot-pressed PEEK implant.

FIG. 22A is the results of Masson staining of hard tissue sections from lateral osseointegration of the rabbit skull 8 weeks after implantation of the hot-pressed PEEK implant.

FIG. 22B is the results of Masson staining of hard tissue sections of the parietal osseointegration of the rabbit skull 8 weeks after implantation of the hot-pressed PEEK implant.

FIG. 22C is a quantitative analysis of the contact ratio of bone implant in the lateral bone of the rabbit skull 8 weeks after implantation of the hot-pressed PEEK implant.

FIG. 22D is a quantitative analysis of bone-implant contact ratios in the parietal bone of the rabbit skull 8 weeks after implantation of the hot-pressed PEEK implant.

FIG. 23 shows a process of S2 of the method for nanomorphological modification of the PEEK surface by hot pressing according to an embodiment of the present application.

### DETAILED DESCRIPTION OF THE EMBODIMENTS

For a better understanding of the technical schemes of the present application by a person of ordinary skill in the art, the technical scheme of the present application is described in further detail below in conjunction with the accompanying drawings and embodiments.

### Embodiment 1

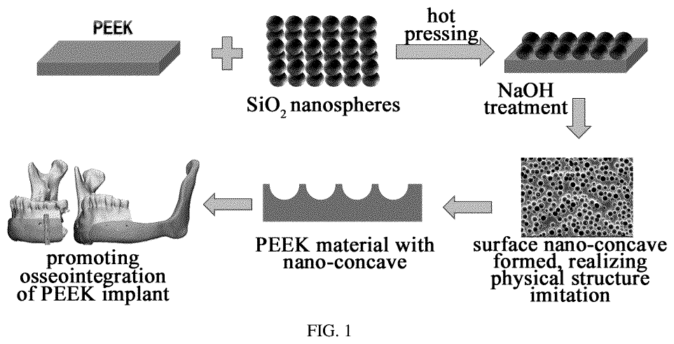

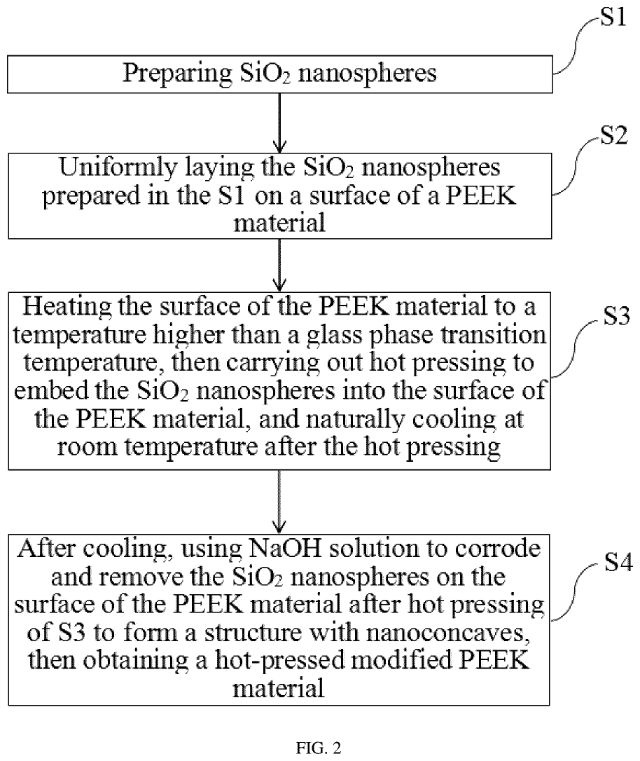

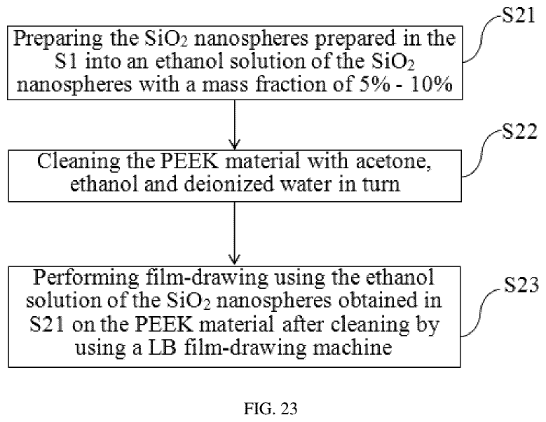

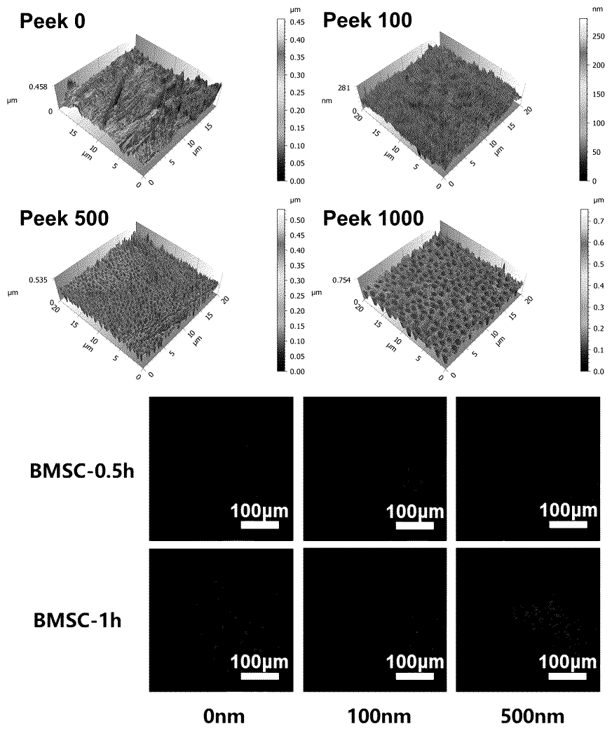

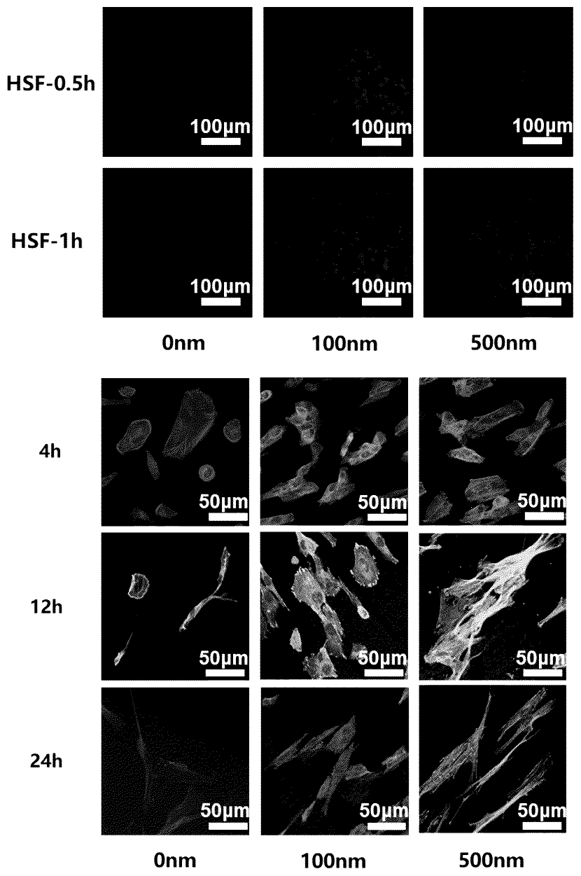

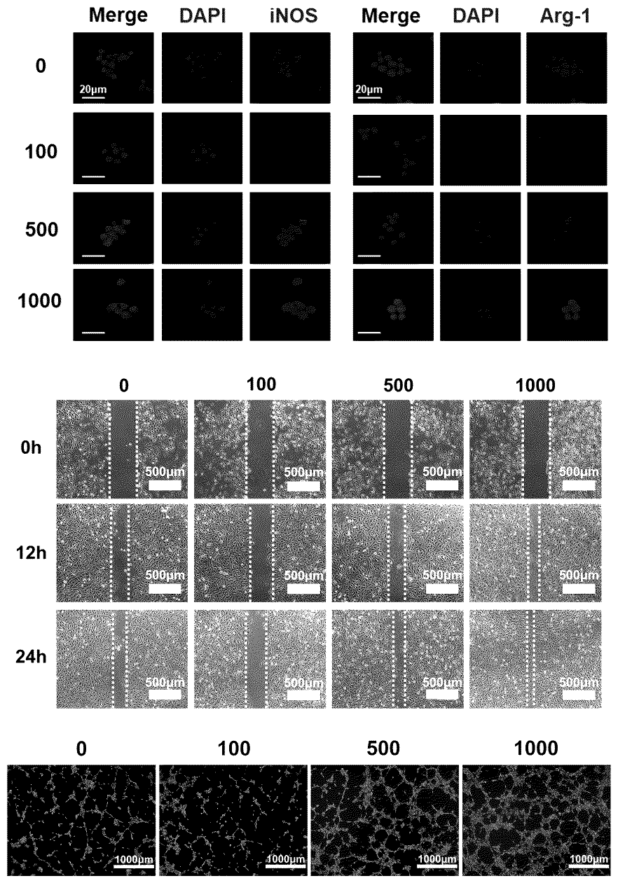

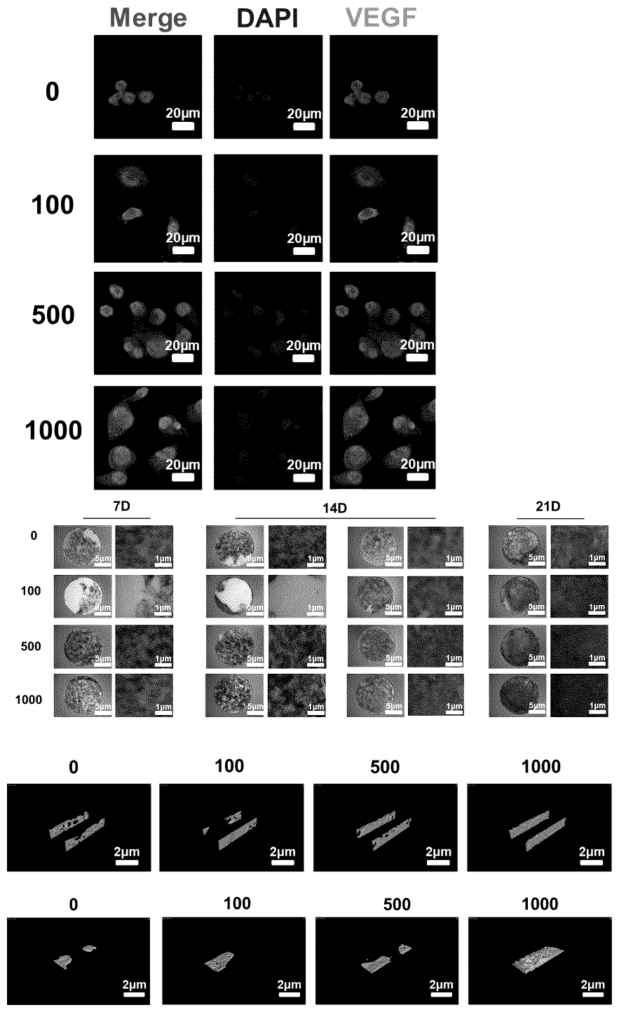

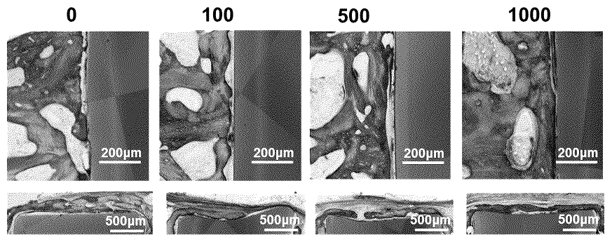

The FIG. 1 and FIG. 2 illustrate a method for nanomorphological modification of polyether ether ketone (PEEK) surface by hot pressing, including the following steps:
S1, preparing SiO₂ nanospheres with a diameter of 100 nanometers (nm);
specifically, ethyl orthosilicate with a mass fraction of 28% is used as a silicon source, aqueous ammonia with a mass fraction of 25%-28% is used as a catalyst, and a sol-gel method is used to prepare monodispersed SiO₂ nanospheres, where a three-necked flask is added with ethanol, water, aqueous ammonia and tetraethyl orthosilicate in a volume ratio of 6:6:1:1, followed by mechanically stirring at 400 revolutions per minute (r/min) and reacting for a duration of 2 hours (h), then centrifuging on a centrifuge at 5,000 r/min to obtain a precipitate; the precipitate is washed 3-4 times with ethanol and baked at 60 degree Celsius (C) for 12 h in an oven to obtain dried SiO₂ nanospheres with the diameter of 100 nm (gradient diameter);
S2, uniformly laying the SiO₂ nanospheres prepared in the S1 on a surface of a PEEK material;
specifically, the obtained SiO₂ nanospheres with the diameter of 100 nm are prepared into an ethanol solution of SiO₂ nanospheres with a mass fraction of 5%; the PEEK material of medical grade is cleaned by acetone, ethanol and deionized water in turn for three times, then a Langmuir-Blodgett (LB) film-drawing machine is used to draw a film on the PEEK material; and
according to FIG. 23, the specific operations of the S2 includes steps as follows:
   S21, preparing the SiO₂ nanospheres prepared in the S1 into an ethanol solution of the SiO₂ nanospheres with a mass fraction of 5%-10%;
   S22, cleaning the PEEK material with acetone, ethanol and deionized water in turn; and
   S23, performing film-drawing using the ethanol solution of the SiO₂ nanospheres obtained in S21 on the PEEK material after cleaning by using a Langmuir-Blodgett (LB) film-drawing machine;
S3, heating the surface of the PEEK material to a temperature slightly higher than a glass phase transition temperature, i.e. 140-160° C., and carrying out hot pressing to embed SiO₂ nanospheres into the surface of the PEEK material;
specifically, the hot pressing is carried out under an air pressure of 0.5 megapascal (MPa) and a hot pressing temperature of 150° ° C. for 15 minutes (min), followed by naturally cooling under room temperature; and
S4, after cooling, using NaOH solution of 5 molality (M) to corrode and remove the SiO₂ nanospheres on the surface of the PEEK material after hot pressing in S3 to form a structure of nanoconcaves with the diameter of 100 nm and depth corresponding to an radius (50 nm), then obtaining a hot-pressed modified PEEK material.

### Embodiment 2

S1, preparing SiO₂ nanospheres with a diameter of 500 nm;
specifically, ethyl orthosilicate with a mass fraction of 28% is used as a silicon source, aqueous ammonia with a mass fraction of 25%-28% is used as a catalyst, and a sol-gel method is used to prepare monodispersed SiO₂ nanospheres, where a three-necked flask is added with ethanol, water, aqueous ammonia and tetraethyl orthosilicate in a volume ratio of 6:6:1:1.5, followed by mechanically stirring at 400 r/min and reacting for a duration of 2 h, then centrifuging on a centrifuge at 5,000 r/min to obtain precipitate; the precipitate is washed 3-4 times with ethanol and baked at 60° C. for 12 h in an oven to obtain dried SiO₂ nanospheres with the diameter of 500 nm;

S2, uniformly laying the SiO₂ nanospheres prepared in the S1 on a surface of a PEEK material;
specifically, the obtained SiO₂ nanospheres with the diameter of 500 nm are prepared into an ethanol solution of SiO₂ nanospheres with a mass fraction of 5%; the PEEK material of medical grade is cleaned by acetone, ethanol and deionized water in turn for three times, then the Langmuir-Blodgett (LB) film-drawing machine is used to draw a film on the PEEK material;

S3, heating the surface of the PEEK material to a temperature slightly higher than a glass phase transition temperature, i.e. 140-160° C., and carrying out hot pressing to embed SiO₂ nanospheres into the surface of the PEEK material;
specifically, the hot pressing is carried out under an air pressure of 0.5 MPa and a hot pressing temperature of 150° C. for 15 min, followed by naturally cooling under a room temperature; and

S4, after cooling, using NaOH solution of 5 M to corrode and remove the SiO₂ nanospheres on the surface of the PEEK material after hot pressing in S3 to form a structure of nanoconcaves with the diameter of 500 nm and depth of 250 nm, then obtaining a hot-pressed modified PEEK material.

### Embodiment 3

S1, preparing SiO₂ nanospheres with a diameter of 1,000 nm;
specifically, ethyl orthosilicate with a mass fraction of 28% is used as a silicon source, aqueous ammonia with a mass fraction of 25%-28% is used as a catalyst, and a sol-gel method is used to prepare monodispersed SiO₂ nanospheres, where a three-necked flask is added with ethanol, water, aqueous ammonia and tetraethyl orthosilicate in a volume ratio of 6:6:3:20, followed by mechanically stirring at 400 r/min and reacting for a duration of 2 h, then centrifuging on a centrifuge at 5,000 r/min to obtain precipitate; the precipitate is washed 3-4 times with ethanol and baked at 60° ° C. for 12 h in an oven to obtain dried SiO₂ nanospheres with the diameter of 1,000 nm;

S2, uniformly laying the SiO₂ nanospheres prepared in the S1 on a surface of a PEEK material;
specifically, the obtained SiO₂ nanospheres with the diameter of 1,000 nm are prepared into an ethanol solution of SiO₂ nanospheres with a mass fraction of 5%; the PEEK material of medical grade is cleaned by acetone, ethanol and deionized water in turn for three times then the film is drawn on PEEK by a LB film-drawing machine;

S3, heating the surface of the PEEK material to a temperature slightly higher than a glass phase transition temperature, i.e. 140-160° C., and carrying out hot pressing to embed SiO₂ nanospheres into the surface of the PEEK material;
specifically, the hot pressing is carried out under an air pressure of 0.5 MPa and a hot pressing temperature of 150° ° C. for 15 min, followed by naturally cooling under room temperature; and

S4, after cooling, using NaOH solution of 5 M to corrode and remove the SiO₂ nanospheres on the surface of the PEEK material after hot pressing in S3 to form a structure of nanoconcaves with the diameter of 1,000 nm and a depth of 500 nm, then obtaining a hot-pressed modified PEEK material.

### Embodiment 4

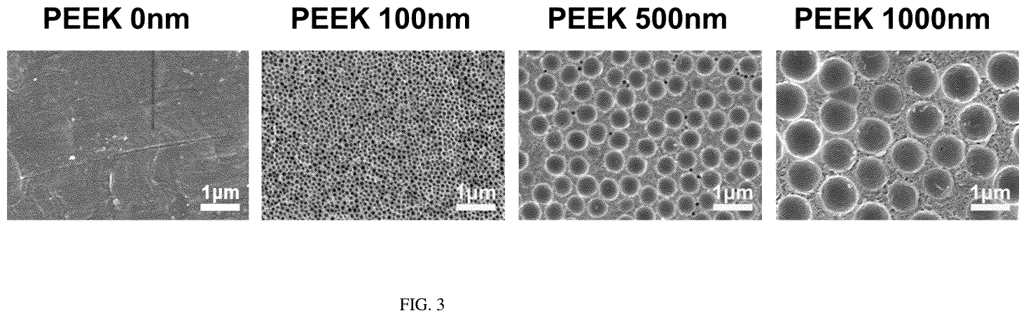

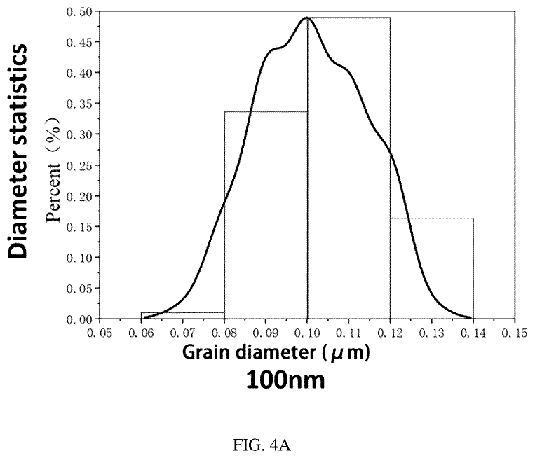

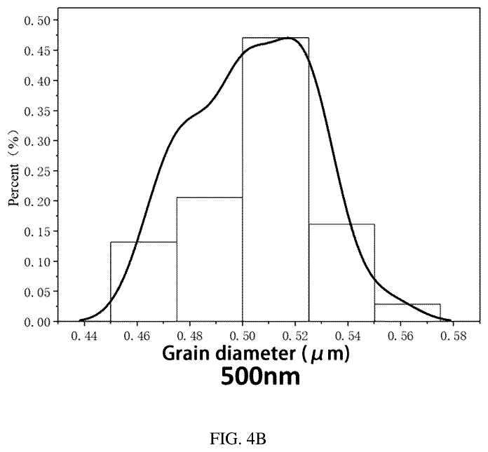

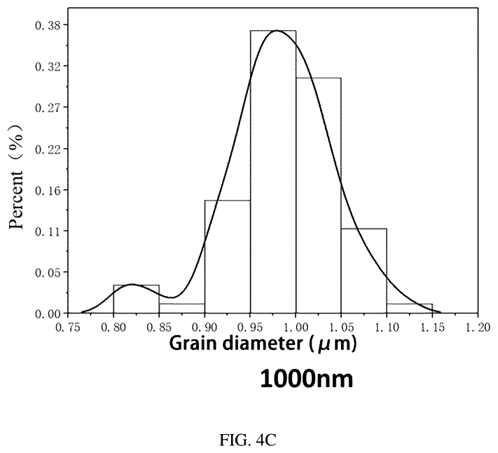

Further, scanning electron microscopy (SEM) is used to observe the PEEK materials prepared in Embodiments 1-3 after hot pressing modification with SiO₂ nanospheres of different diameters. FIG. 3 and FIG. 4A-FIG. 4C show the SEM images of the surface of the hot-pressed modified PEEK materials (after NaOH treatment) and the corresponding nanoconcaves diameters statistics, respectively. From FIG. 3 and FIG. 4A-FIG. 4C, it can be seen that hot-pressed nanoconcaves morphologies with diameters of 100, 500, and 1,000 nm are successfully constructed on the surface of the hot-pressed nanoconcaves group compared with that of pure PEEK.

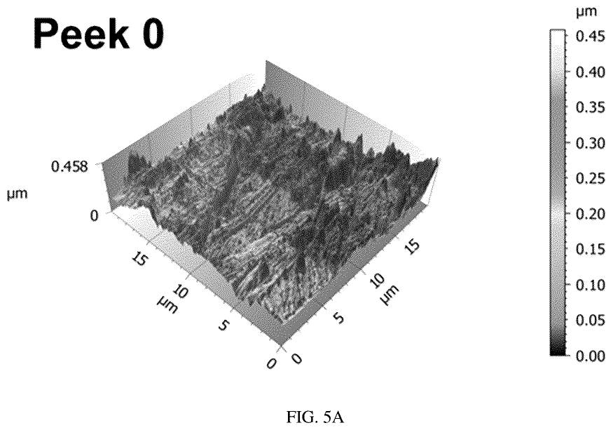

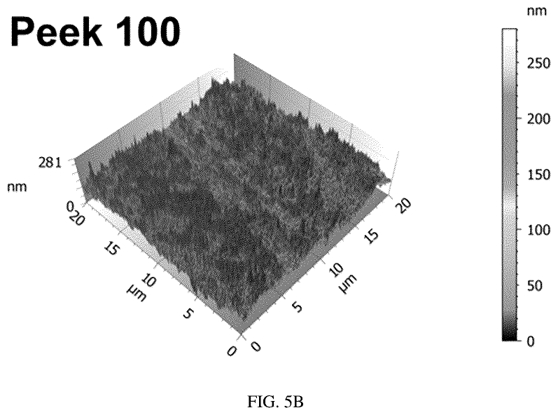

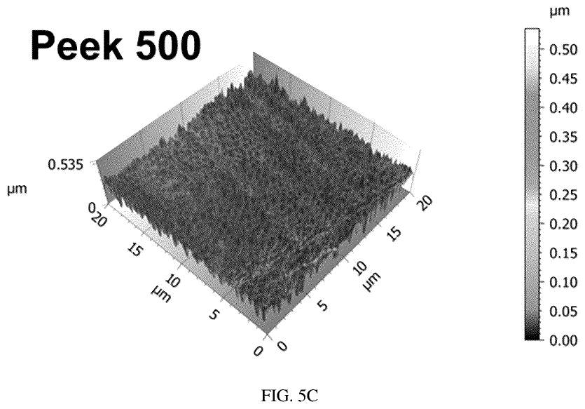

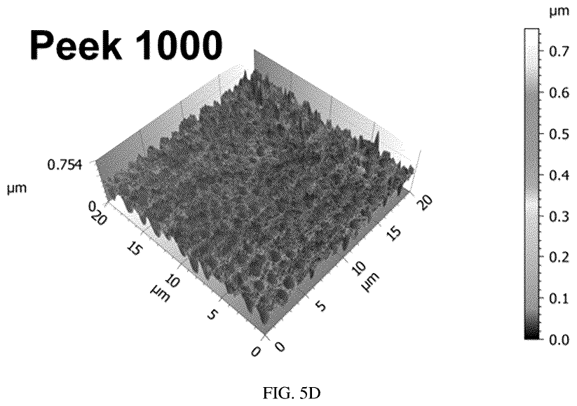

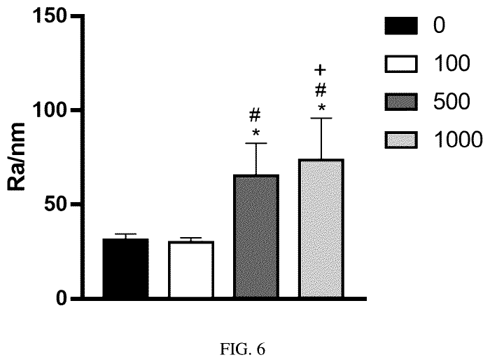

AFM is used to observe the 3D microscopic morphology of each group of materials and to quantitatively detect the roughness. The AFM images of FIG. 5A-FIG. 5D show that there are traces of machining and sanding on the surface of the blank PEEK, and dense nanopores are observed on the surface of the material in the 100 nm group, and obvious pit-like microstructures matching the respective diameters are observed in the 500 nm and 1000 nm groups. The roughness test is shown in FIG. 6, and the results of that of 100 nm, 500 nm, and 1000 nm pressed pitted PEEK surfaces demonstrate that the roughness of each group of materials increases with the increase of the diameter of the nano-pressed pits.

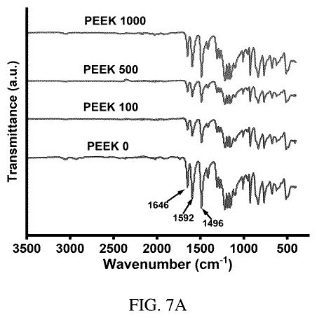

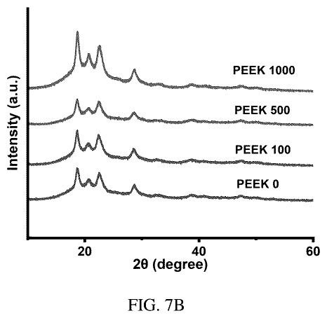

Fourier transform infrared spectroscopy (FTIR) and X-ray diffraction (XRD) are used to detect the element change and phase transformation of PEEK materials modified by hot pressing with SiO₂ nanospheres with different diameters. FIG. 7A-FIG. 7B show the Fourier infrared detection and x-ray diffraction detection of the surface of the hot-pressed modified PEEK materials, respectively;
the mechanical properties of each group of materials are tested by nanoindentation instrument for any changes. The statistical results of FIG. 7A-FIG. 7B show that there is no significant change in the modulus of elasticity as well as nano-hardness between the groups of materials, suggesting that the nano press-pitting technique does not affect the mechanical properties of the material substrate, and that the effect of factors such as hardness on the subsequent in vitro cells as well as in vivo tissues can be ruled out.

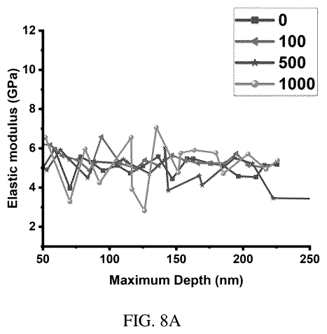

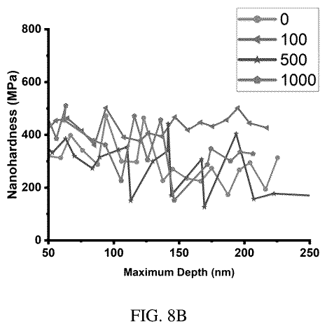

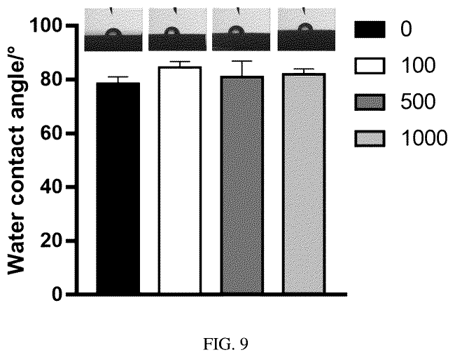

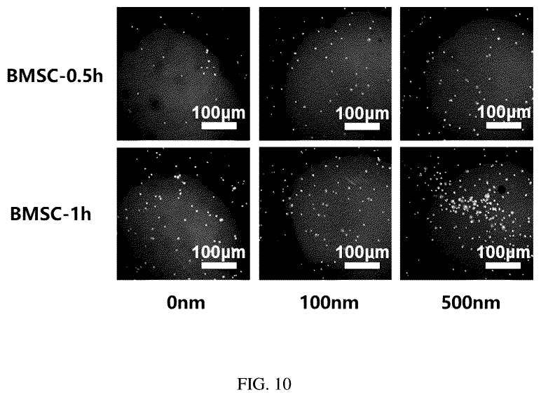

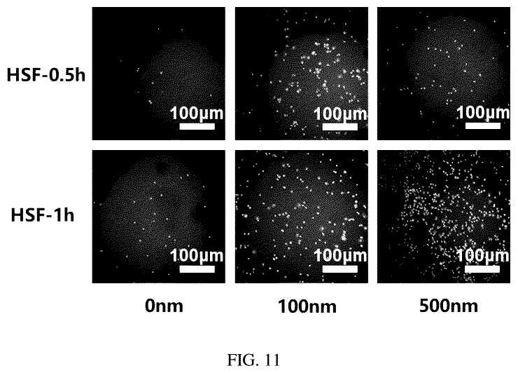

The difference in the wettability of each group of materials is measured using a contact angle meter. As shown in FIG. 8A-FIG. 8B, the water contact angle of the 100 nm group is slightly higher, yet no statistically significant difference is found compared with the other groups, which proves that the materials of each group treated by hot pressing are basically the same as pure PEEK, and the hydrophilicity has no significant change, confirming that the hot pressing treatment does not affect the hydrophilicity, and that the effect of factors such as wettability on the subsequent in vitro cells as well as in vivo tissues can be ruled out.

As can be seen from FIG. 5A-FIG. 8B, only the surface morphology of the PEEK materials after hot pressing of SiO₂ nanospheres of different diameters is transformed, suggesting that the influence of elements as well as physical phases, mechanical properties and wettability is excluded.

### Embodiment 5

The hot-pressed modified PEEK materials prepared in Embodiments 1-3 are used as bone implants in bone implantation.

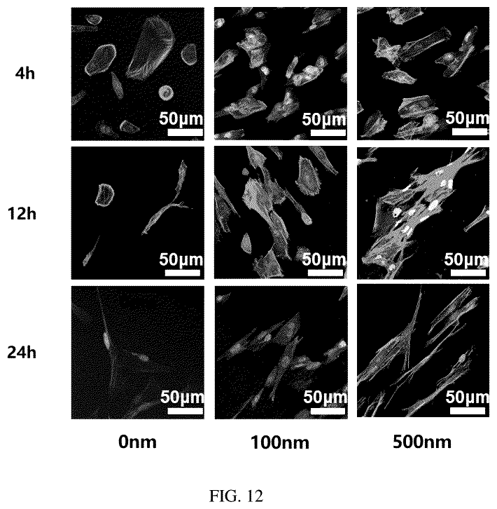

Specifically, rat bone marrow stromal cells (rBMSCs) as well as human skin fibroblast (HSF) cells are cultured on the surface of the hot-pressed modified PEEK material using an in vitro cellular assay, and the adhesion effect of the hot-press modified PEEK material to the cells is tested by 4′,6-diamidino-2′-phenylindole (DAPI) staining. FIG. 10 and FIG. 11 show the observation diagrams of DAPI staining of rBMSCs and HSF cells cultured on the surface of hot-pressed modified PEEK material, respectively, and it can be seen from the drawings that the cell adherence of rBMSCs and HSF cells in the 500 nm group is significantly greater than that in the pure PEEK group.

HSF cells are cultured on the surface of hot-pressed modified PEEK material, and their adhesion-related protein expression is observed by immunofluorescence of adhesion plaque protein vinculin. As can be seen from FIG. 12, the immunofluorescence observation of HSF cells cultured on the surface of hot-pressed modified PEEK material, the expression of vinculin in the 500 nm group is obvious and the cell morphology is more stretched.

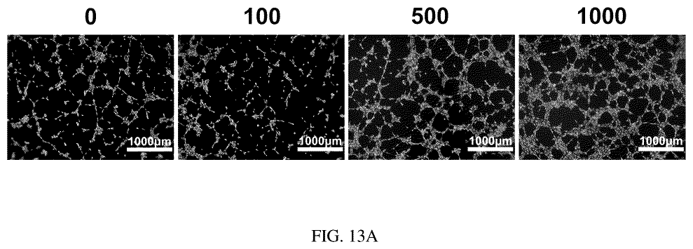

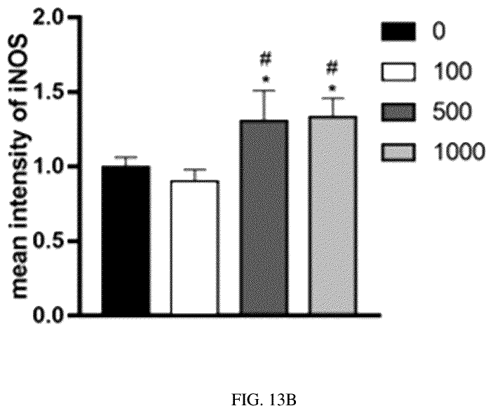

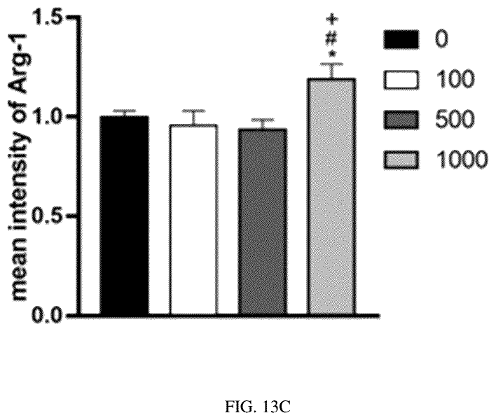

After inoculation of macrophages on the surface of each group of materials for 6 h, immunofluorescence staining of iNOS and Arg-1 is performed to detect the effects of the material surface on the M1 and M2 polarization of macrophages, respectively. After inoculation of macrophages on the surface of each group of materials for 6 h, immunofluorescence staining of iNOS and Arg-1 is performed to detect the effects of the material surface on the M1 and M2 polarization of macrophages, respectively. iNOS immunofluorescence quantification according to FIG. 13A-FIG. 13C shows that the 500 nm and 1000 nm groups can promote the M1 polarization of macrophages at 6 h, the quantitative results of Arg-1 immunofluorescence also show that the 1000 nm group can promote the M2 polarization of macrophages at 6 h.

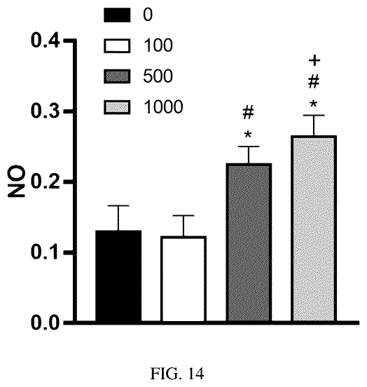

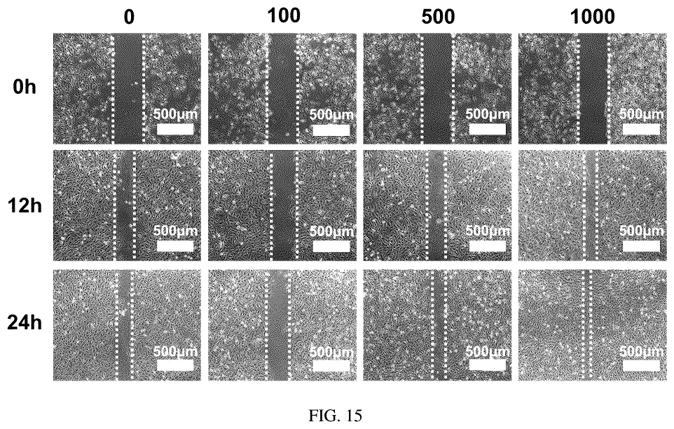

The ability to promote angiogenesis is assessed by detecting the NO release from macrophages cultured on the surface of 0, 100 nm, 500 nm, and 1000 nm pressed-pitted PEEK surface materials after 6 h. The results are shown in FIG. 14, the results of NO release from macrophages cultured on 0, 100 nm, 500 nm, and 1000 nm pressed-pitted PEEK surfaces indicate that the material surfaces of the 500 nm and 1000 nm groups have better angiogenesis-promoting abilities than those of the pure PEEK group, whereas the material surfaces of the 100 nm group have lower pro-angiogenic abilities than those of the pure PEEK group.

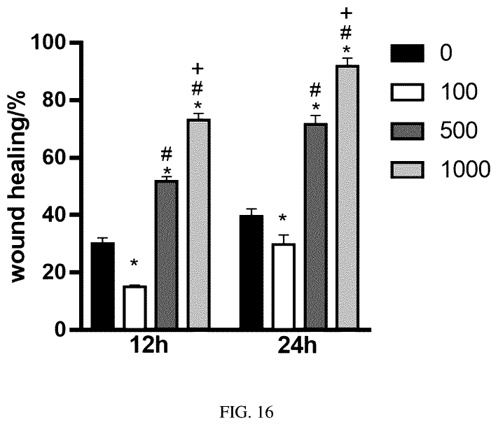

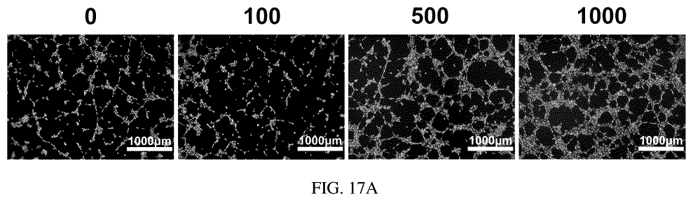

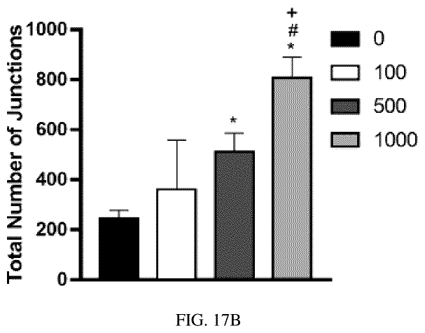

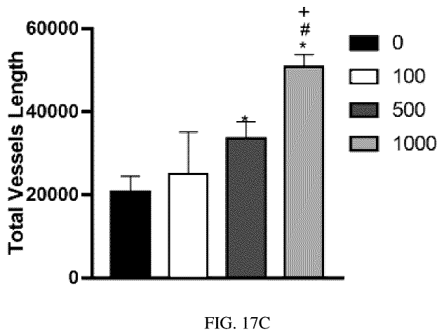

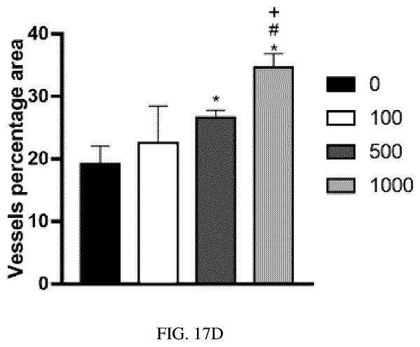

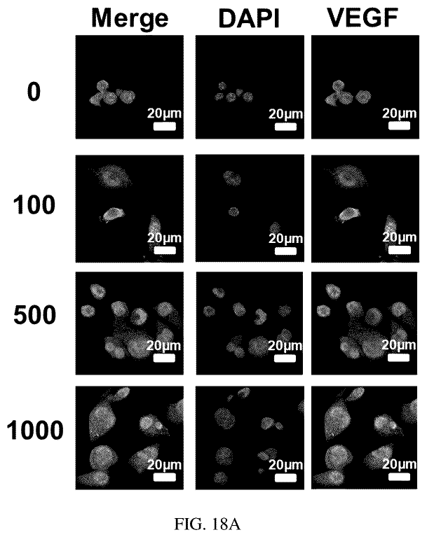

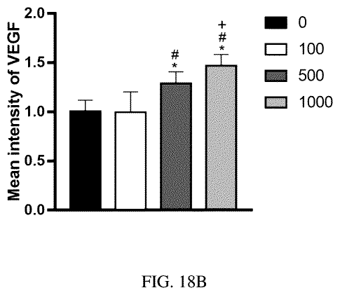

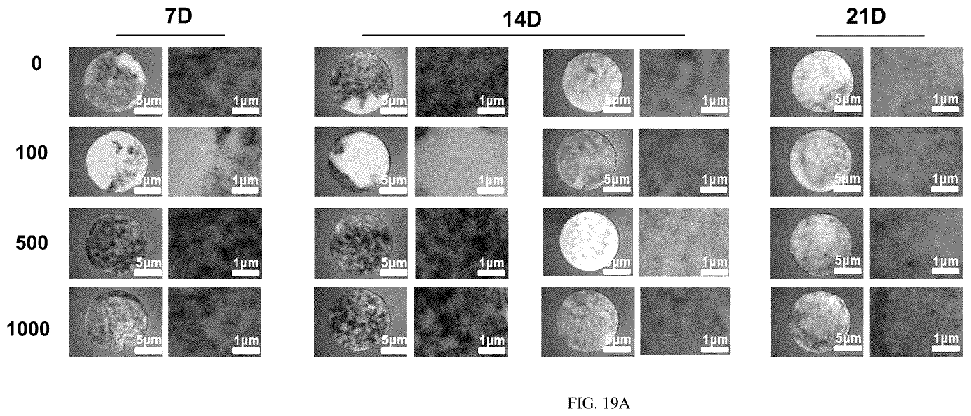

The vasculogenic effect of the surface of hot-pressed modified PEEK material on HUVECs is examined by scratching experiments, lumen formation experiments and VEGF immunofluorescence staining. As shown in FIG. 15, the results of the HUVECs scratching experiment show that the 500 nm and 1000 nm groups can promote the migration of HUVECs and achieve a significant healing effect at 12 h and 24 h. The healing effect of the 0 and 100 nm groups is not significant. Statistical results as shown in FIG. 16 demonstrate that the scratch healing rates of the 500 and 1000 groups are significantly higher than those of the 0 and 100 nm groups, and the scratch healing rate of the 1000 nm group is slightly higher than that of the 500 nm group. As shown in FIG. 17A-FIG. 17D, the results of in vitro vessel-forming experiments of HUVEC suggest that the 500 nm and 1000 nm groups can promote blood vessel formation, and obvious tubular structures are formed by HUVECs at 4h, while those in the 0 and 100 nm groups are mainly scattered and discontinuous branch-like structures. The statistical results suggest that the total number of vessel branches, total vessel length, and total vessel area in the 1000 nm group are better than those in the other three groups, the 500 nm group is better than the 0 group, and there is no significant difference between the 0 and 100 nm groups. As shown in FIG. 18A-FIG. 18B, the results of VEGF immunofluorescence assay indicate that the 500 nm group has the highest expression of VEGF, and the 1000 nm group has a slightly weaker expression, both of which are stronger than those of the 100 nm group and the pure PEEK group. As can be seen from the results of fluorescence quantification, the fluorescence intensity of each group is ordered from high to low as: 500 nm>1000 nm>100 nm>0 nm.

The vasculogenic effect of the surface of hot-pressed modified PEEK material on HUVECs is examined by scratching experiments, lumen formation experiments and VEGF immunofluorescence staining. As shown in FIG. 15, the results of the HUVECs scratching experiments indicate that the 500 nm and 1000 nm groups can promote the migration of HUVECs at 12h and 24h, and the healing effect is significant. The healing effect in the 0 and 100 nm is not obvious. Statistical results as shown in FIG. 16 indicate that the scratch healing rates of the 500 and 1000 groups are significantly higher than those of the 0 and 100 nm groups, and the scratch healing rate of the 1000 nm group is slightly higher than that of the 500 nm group. As shown in FIG. 17A-FIG. 17D, the results of in vitro vessel-forming experiments of HUVEC indicate that the 500 nm and 1000 nm groups can promote blood vessel formation, and obvious tubular structures are formed by HUVECs at 4 h, while those in the 0 and 100 nm groups are mainly scattered and discontinuous branch-like structures. Statistical results show that the total number of vascular branches, the total length of blood vessels, and the total area of blood vessels in the 1000 nm group are superior to the other three groups, the 500 nm group is superior to the 0 group, and there is no significant difference between the 0 and 100 nm groups. As shown in FIG. 18A-FIG. 18B, the results of VEGF immunofluorescence assay indicate that the 500 nm group has the highest expression of VEGF, and the 1000 nm group has a slightly weaker expression, both of which are stronger than the 100 nm group and the pure PEEK group. according to the fluorescence quantification results, the fluorescence intensity of each group is ranked from high to low: 500 nm>1000 nm>100 nm>0 nm.

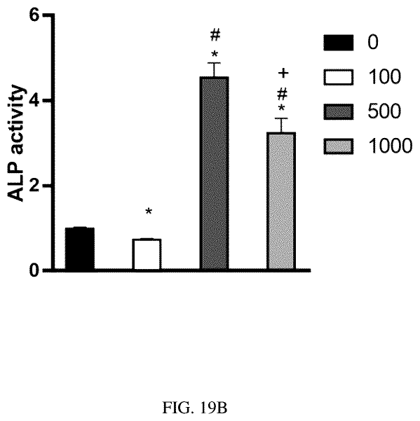

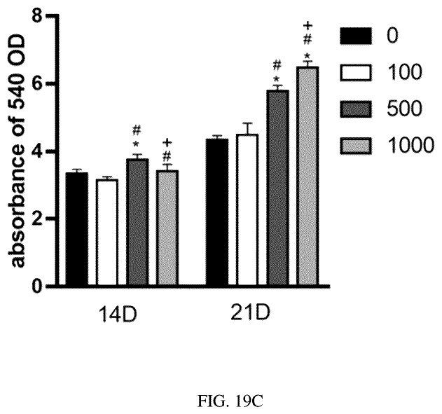

The osteogenic properties of PEEK modified by hot pressing with SiO₂ nanospheres of different diameters are examined in vitro, whereas the indexes of in vitro osteogenic differentiation of PEEK modified by hot pressing are shown in FIG. 19A-FIG. 19C, including alkaline phosphatase (ALP) and alizarin red staining, where ALP (a marker of early osteogenesis) on days 7 and 14, and alizarin red staining (to detect calcium salt deposition) on days 14 and 21 are measured, respectively; the staining results show that the ALP as well as the alizarin red staining at each time point in the 500 nm and 1,000 nm groups are darker and wider compared to the pure PEEK (0 nm) and 100 nm groups, all with good in vitro osteogenic properties.

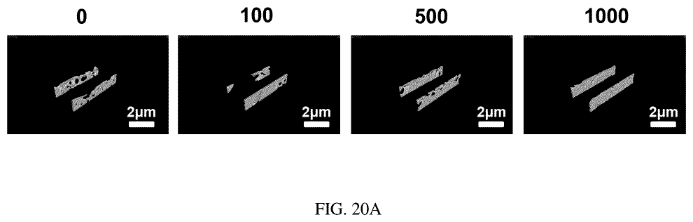

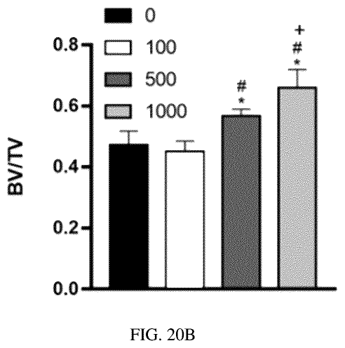

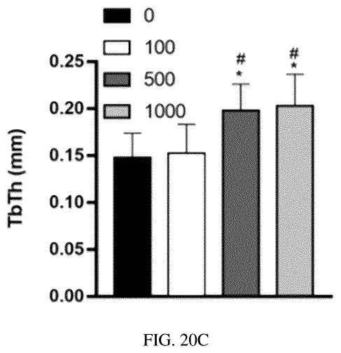

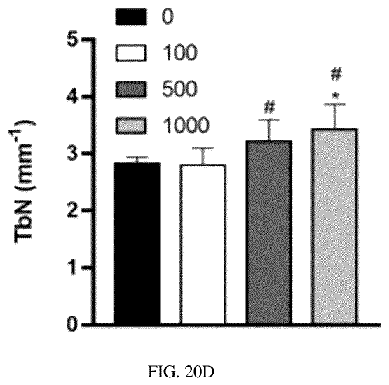

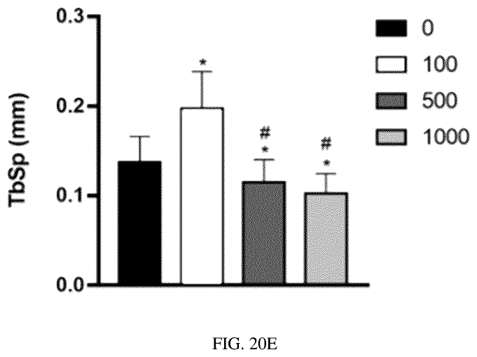

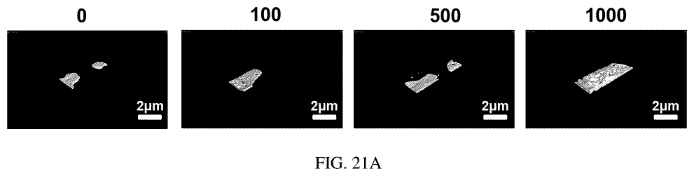

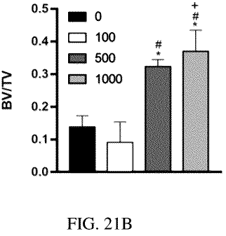

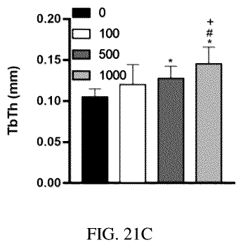

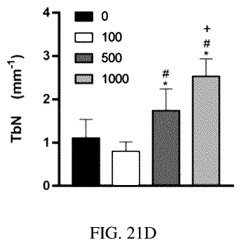

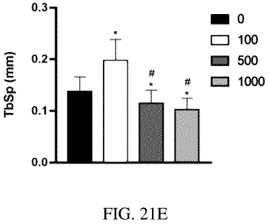

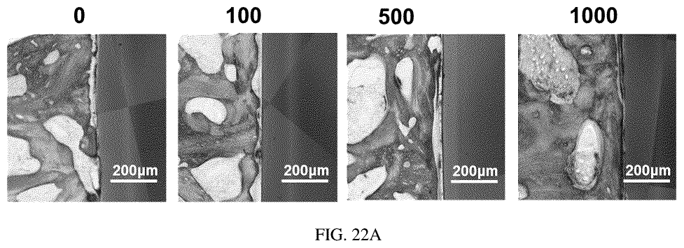

In vivo implantation experiments are conducted in the rabbit cranium for the implantation of thermocompression-modified PEEK materials, which are divided into four groups, namely, pure PEEK, 100 nm, 500 nm, and 1000 nm, and the four groups of materials are removed one month after implantation; micro-CT is performed to assess the generation of new bone around the PEEK implant, and the area is divided into the PEEK implant, the osteoconductive zone of lateral bone tissue contact, and the osteoinductive zone of the parietal surface; the detection indexes include BV/TV (bone tissue/total volume ratio), TbTh (trabecular thickness), TbN (trabecular number), and TbSp (trabecular spacing). FIG. 20A-FIG. 20E and FIG. 21A-FIG. 21E are micro-CT observations of osteogenesis on the lateral and parietal surfaces of the implant after cranial implantation of modified PEEK implants in rabbits, respectively. Compared with that of the pure PEEK group (0), the peri-implant of the 500 and 1000 nm groups has higher BV/TV, TbTh, and TbN, as well as smaller TbSp, which proves that the volume of new bone (BV/TV) as well as bone quality (TbTh and TbN) around the implants in 500 and 1000 groups are better than that of the pure PEEK group (0). This demonstrates that both the 500 and 1000 groups of implants can better promote the generation of bone tissue around the implant. The difference in the osteogenesis of 500 and 1000 implants compared to 0 and 100 implants is greater on the top surface than on the side surface, which demonstrates that the implants have a great advantage in inducing osteogenesis.

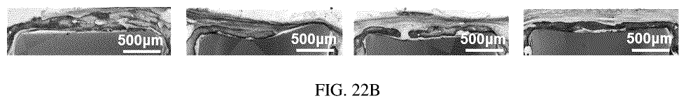

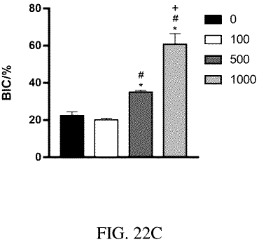

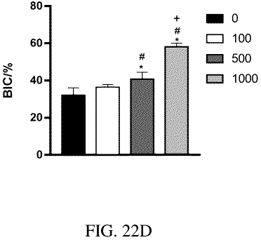

Masson staining of hard tissue sections from the four groups is performed to assess the osseointegration of the implants. Osteointegration is the “gold standard” for implant substitution and force-bearing after implant placement, referring to the direct contact between the implant and the surrounding bone tissue without obvious fibrous tissue encapsulation. FIG. 22A-FIG. 22D shows the Masson staining observation of hard tissue sections of modified PEEK implant rabbit skulls after implantation and the corresponding quantitative statistics, from which it can be seen that the 500 and 1000 nm groups have higher direct bone tissue contact in both lateral and parietal surfaces, and the 1000 group is superior to the 500 group. Quantitative statistics indicate that the BIC (bone implant contact) of the 500 and 1000 groups is significantly higher than that of the pure PEEK group (0) and the 100 nm group, and that of the 1000 group is higher than that of the 500 group; therefore, the 1000 and 500 groups have better osseointegration properties, and the osteointegration effect of the 1000 group is the best.

Conclusion: the purely morphological modification of nanoconcaves on the PEEK surface by hot pressing method involves no elemental or physical changes, and promotes its early vascularisation after implantation, osteogenesis as well as osseointegration in vivo only through the modification of nanomorphology, with results showing that the treatment with nanoconcaves of 500 and 1,000 nm diameter is more effective in promoting osseointegration. The method of surface modification involved in the present application offers the advantages of integrated safety, practicality and effectiveness, and has great potential for clinical application.

The above illustrates and describes the basic principle of the present application, as well as the main features and the advantages of the present application. It is to be understood by those skilled in the art that the present application is not limited by the above embodiments, and that what is described in the above embodiments and the specification only illustrates the principle of the present application. Without departing from the spirit and scope of the present application, there are various variations and improvements to the present application, all of which fall within the scope of the present application claimed for protection. The scope of protection claimed for the present application is defined by the appended claims and their equivalents.

What is claimed is:

1. A method for nanomorphological modification of a polyether ether ketone (PEEK) surface by hot pressing, comprising:
S1, preparing SiO₂ nanospheres;
S2, uniformly laying the SiO₂ nanospheres prepared in the S1 on a surface of a PEEK material;
S3, heating the surface of the PEEK material to a temperature higher than a glass phase transition temperature, then carrying out hot pressing to embed the SiO₂ nanospheres into the surface of the PEEK material, and naturally cooling at a room temperature after the hot pressing; and
S4, after the cooling, using NaOH solution to corrode and remove the SiO₂ nanospheres on the surface of the PEEK material after the hot pressing in the S3 to form a structure with nanoconcaves, and then obtaining a hot-pressed modified PEEK material.

2. The method for nanomorphological modification of a PEEK surface by hot pressing according to claim 1, wherein the S1 comprises specific operations as follows:
using ethyl orthosilicate as a silicon source and aqueous ammonia as a catalyst, and preparing monodispersed SiO₂ nanospheres by a sol-gel method, wherein the SiO₂ nanospheres are in a diameter of 100-1,000 nanometers.

3. The method for nanomorphological modification of a PEEK surface by hot pressing according to claim 2, wherein a mass fraction of the ethyl orthosilicate is 28%, a mass fraction of the aqueous ammonia is 25%-28%; and the sol-gel method comprises adding ethanol, water, the aqueous ammonia and tetraethyl orthosilicate in a volume ratio of 6:6:1:(1-10), and reacting for a duration of 2-24 hours.

4. The method for nanomorphological modification of a PEEK surface by hot pressing according to claim 3, wherein specific operations of the S2 comprises steps as follows:
S21, preparing the SiO₂ nanospheres prepared in the S1 into an ethanol solution of the SiO₂ nanospheres with a mass fraction of 5%-10%;
S22, cleaning the PEEK material with acetone, ethanol and deionized water in turn; and
S23, performing film-drawing using the ethanol solution of the SiO₂ nanospheres obtained in the S21 on the PEEK material after cleaning by using a Langmuir-Blodgett film-drawing machine.

5. The method for nanomorphological modification of a PEEK surface by hot pressing according to claim 4, wherein in the S3, a temperature for the hot pressing is 140-160 degrees Celsius, an air pressure of the hot pressing is 0.5-0.8 megapascal, and a duration for the hot pressing is 15-20 minutes.

6. The method for nanomorphological modification of a PEEK surface by hot pressing according to claim 5, wherein a concentration of the NaOH solution in the S4 is 5-7 molality.

7. A hot-pressed modified PEEK material obtained by the method for nanomorphological modification of a PEEK surface by hot pressing according to claim 1.

8. The hot-pressed modified PEEK material according to claim 7, wherein a structure with nano-concave on a surface of the hot-pressed modified PEEK material has a diameter of 100-1,000 nanometers and a depth of 50-500 nanometers.

9. An application of the hot-pressed modified PEEK material according to claim 7 as a bone implant.
